(19) United States
(12) Patent Application Publication
(10) Pub. No.: US 2020/0000631 A1
(43) Pub. Date: Jan. 2, 2020

(54) HOT AND COLD PACK WITH A CLOSABLE POUCH

(71) Applicant: YSCS Products, LLC, Southfield, MI (US)

(72) Inventors: Sarah Herzka, Lakewood, NJ (US); Yocheved Shifra Tannenbaum, Southfield, MI (US)

(21) Appl. No.: 16/019,841

(22) Filed: Jun. 27, 2018

(51) Int. Cl. A61F 7/02

(52) U.S. Cl. CPC A61F 7/02; A61F 2007/0219; A61F 2007/0028; A61F 2007/0222; A61F 2007/0223

(57) ABSTRACT

An apparatus is provided. The apparatus may include a gel pack. The gel pack may contain the gel filling. The gel filling may measure between about 110-250 grams. The apparatus may include a top cover and a bottom cover. The top cover and the bottom cover may be comprised of nylon and PVC. The top cover and the bottom cover may measure between about 22-26 centimeters long and between about 6.5-8.5 centimeters wide, when measured flat. The gel pack may be frozen and/or heated.

### FIELD OF THE DISCLOSURE

This disclosure relates to packs. More specifically, this disclosure relates to hot and cold packs.

### BACKGROUND OF THE DISCLOSURE

Conventionally, packs are used for cold and/or heat therapy for a variety of different ailments. Packs are also used to soothe and/or relax sensitive skin.

### SUMMARY OF THE DISCLOSURE

A perineal-sized apparatus is provided. The apparatus may include a gel pack. The gel pack may include a gel filling. The apparatus may include a gel pack. The apparatus may include a closable pouch. The closable pouch may be for partial and/or total enclosure of the gel pack. When the gel pack is frozen to at least less than about twelve degrees Fahrenheit, the gel filling may maintain a temperature under 60 degrees Fahrenheit after being placed in skin contact for a minimum of 27 minutes.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows a perspective view of an illustrative gel pack according to embodiments of the invention;

FIG. 2 shows a side view of the long side of an illustrative gel pack according to embodiments of the invention;

FIG. 3 shows a side view of the short side of an illustrative gel pack according to embodiments of the invention;

FIG. 4 shows a top view of a gel pack according to embodiments of the invention;

FIG. 5 shows a perspective view of an illustrative gel pack and an illustrative pouch according to embodiments of the invention;

FIG. 6 shows an illustrative gel pack fully enclosed by an illustrative pouch according to embodiments of the invention;

FIG. 7 shows an illustrative gel pack partially enclosed by an illustrative pouch according to embodiments of the invention;

FIG. 8 shows an illustrative drawstring bag according to embodiments of the invention;

FIG. 9 shows an illustrative set including a drawstring bag, three gel packs and three pouches according to embodiments of the invention;

FIG. 10 shows an illustrative graph showing temperature changes according to embodiments of the invention;

FIG. 11 shows another illustrative graph showing temperature changes according to embodiments of the invention;

FIG. 12 shows yet another illustrative graph showing temperature changes according to embodiments of the invention;

FIG. 13 shows still another illustrative graph showing temperature changes according to embodiments of the invention;

FIG. 14 shows yet another illustrative graph showing temperature changes according to embodiments of the invention;

FIG. 15 shows still another illustrative graph showing temperature changes according to embodiments of the invention;

FIG. 16 shows yet another illustrative graph showing temperature changes according to embodiments of the invention; and

FIG. 17 shows still another illustrative graph showing temperature changes according to embodiments of the invention.

### DETAILED DESCRIPTION OF THE DISCLOSURE

An apparatus is provided. The apparatus may include a gel filling. The apparatus may include a top cover and a bottom cover. The top cover and/or the bottom cover may measure between about 22-26 centimeters long and between about 6.5-8.5 centimeters wide, when measured flat. In some embodiments, the top cover and/or the bottom cover may measure between about 23-24 centimeters long and between about 7.2-7.8 centimeters wide, when measured flat. In some embodiments, the top cover and/or the bottom cover may measure about 23.6 centimeters long and 7.6 centimeters wide.

In some embodiments, the top cover and/or the bottom cover may each include a multilayer material. The multilayer material may be pressed together. The multilayer material may include a layer of nylon and/or a layer of polyvinyl chloride (“PVC”). In some embodiments, the thickness of the top cover and/or bottom cover may be between about 0.1 and 0.5 millimeters. In some embodiments, the thickness of the top cover and/or bottom cover may be 0.3 millimeters. The nylon layer of the cover may be 0.1 millimeters thick. The PVC layer of the cover may be 0.2 millimeters thick. The nylon and PVC may be combined to create the material used for the top cover and/or bottom cover.

In some embodiments, the PVC side of the material may face the gel filling. Therefore, the top cover may be positioned in such a way that the nylon layer is facing away from the gel and the PVC layer is facing (and in fact touching) the gel filling. The bottom cover may be positioned in such a way that the nylon layer is facing away from the gel and the PVC layer is facing (and in fact touching) the gel filling.

In some embodiments, the gel filling may be between about 100 grams and 250 grams. Exemplary weights of the gel filling may include ranges of between about 110 grams and 250 grams, between about 120 grams and 200 grams, between about 130 grams and 170 grams, between about 140 grams and 160 grams, between about 150 grams and 170 grams and between about 150 grams and 160 grams. In some embodiments, the gel filling may weigh 150 grams.

In some embodiments, the weight of the gel filling may be measured by the concentration of the gel filling per square centimeter of the top cover and/or bottom cover measured flat. In these embodiments, the gel filling may be between about 661.10735481932 and 1,202.0133723988 milligrams per centimeter squared. In some embodiments, the gel filling may be between about 836.3 and 1,115.1 milligrams per centimeters squared. In some embodiments, the gel filling may be 975.7 milligrams per centimeters squared. It should be appreciated that these milligrams per centimeter squared measurements may include a sealed edge, of which the measurements are described below, that does not include the gel filling.

In some embodiments, the gel filling may be comprised of a combination of glycerin, carboxymethyl cellulose (“CMC”) and water. The water may be purified water. Exemplary percentages of the combination may include between about 5-25 percent glycerin, between about 60-90 percent CMC and between about 3-25 percent water, between about 10-20 percent glycerin, between about 70-80 percent CMC and between about 5-15 percent water, between about 12-18 percent glycerin, between about 70-80 percent CMC and between about 5-15 percent water, about 15 percent glycerin, about 75 percent CMC and about 10 percent water.

In some embodiments, the top cover and the bottom cover may be sealed together to form a sealed gel pack. The sealing may be accomplished by a high frequency sealing method. The high frequency sealing method may seal an edge around the gel pack. The width of the sealed edge may be between about 1 millimeters to 10 millimeters. In some embodiments, the sealed edge may be between about 4 millimeters and 8 millimeters. In some embodiments, the sealed edge may be about 5 millimeters. 5 millimeters may be a sufficiently strong, reinforced edge to ensure that when the weight of a person sitting on the gel pack does not cause the gel pack to break open. In some embodiments, the edge of the gel pack may be a soft edge. The soft edge may reduce un-comfortabilities when handling the gel pack. The gel pack may contain the gel filling.

In other embodiments, the gel pack may be created from a bag-like structure. In these embodiments, the bag-like structure may also enclose the gel filling. In these embodiments, a top cover and/or a bottom cover may not be included.

The gel pack may be frozen. The temperature of the frozen gel pack may be less than 32 degrees Fahrenheit. The temperature of the frozen gel pack may be less than twelve degrees Fahrenheit. The temperature of the frozen gel pack may be less than two degrees Fahrenheit. The temperature of the frozen gel pack may be less than zero degrees Fahrenheit. The gel pack may be frozen in a typical home freezer. The gel pack may be frozen in a commercial freezer. The gel pack may be frozen in any other suitable manner. In some embodiments, the gel pack may be cooled and not frozen. The gel pack may preferably remain flexible even in the event that the gel is frozen.

The gel pack may be placed in a location that does not contribute to maintaining a frozen temperature. Such a location may be in direct or indirect contact with live human or animal skin. Such a location may include a perineal area, rectal area, vaginal area, genital area, perineum, scrotum and/or anus of a human. For the purposes of this application, the perineal area may include one or more of the above-mentioned body parts. In some embodiments, the gel pack may maintain a temperature under sixty degrees Fahrenheit after being placed in such a location (referred to herein as adjacent to perineal area) for a minimum of 27 minutes. Table A, below, shows exemplary temperatures and predetermined times. Each of the points represents a point in time at which the given is temperature is higher than a temperature associated with device according to certain embodiments and lower than a temperature associated with a prior art device. Additionally, it should be noted that all temperature measurements in this application have been measured in Fahrenheit unless otherwise noted.

TABLE A

| Temperature | After being placed in skin |
|---|---|
| (Fahrenheit), under which | contact for amount of |
| the gel filling is maintained | minutes shown below: |
| 14° F. | 2 |
| 20° F. | 4 |
| 34° F. | 9 |
| 46° F. | 14 |
| 36° F. | 18 |
| 41° F. | 31 |
| 50° F. | 20 |
| 52° F. | 31 |
| 56° F. | 22 |
| 60° F. | 26 |
| 64° F. | 31 |
| 60° F. | 32 |

The apparatus may also include a closable pouch. The closable pouch may be for partial and/or total enclosure of the gel pack. The closable pouch may be constructed of disposable fabric. The disposable fabric made include and/or be constructed of one or more of: polypropylene, Medical SMS polypropylene, cotton, plastic, tri-laminate construction and/or laminate construction. The disposable fabric may be any suitable thickness. The closable pouch may be constructed of washable and/or reusable fabric. Such a fabric may include microfiber, terry cloth, cotton cloth or any other suitable fabric. Such a fabric may be made from cotton and polyester blend. In some embodiments, the percentage of cotton included in a cotton and polyester blend may include between about 70-100% cotton and 0-30% polyester. In some embodiments, the percentage of cotton may include about 80% cotton and 20% polyester.

In some embodiments, the thickness of the fabric may be between about 100-300 grams per square meter (“gsm”). In some embodiments, the thickness of the fabric may be between about 150-200 gsm.

In one exemplary preferred embodiment, the fabric may be terry cloth fabric made from 80% cotton and 20% polyester weighing between about 150-200 gsm.

The color of the pouch and/or gel pack may be black, red, purple, white or any other suitable color. In one preferred embodiment, the color of the pouch may be partially and/or completely black. In this embodiment, the black color may hide any stains or soil spots that may discolor or otherwise reduce the appeal of the pouch or gel pack.

The pouch may include a flap. When the flap is in a closed position, the pouch may completely enclose the gel pack. It should be appreciated that complete enclosure of the gel pack is beneficial because the fabric pouch is soft on sensitive skin. If the pouch does not completely enclose the gel pack, the gel pack may fall out of the pouch during normal human movement. The gel pack may be excessively cold and/or hot to the skin without the protective barrier of the pouch. Therefore, complete enclosure of the gel pack provides a protective layer that protects sensitive skin in any position the human moves.

It should also be appreciated that, in some embodiments, the pouch may be machine-washable. In these embodiments, the pouch may become soiled during normal use. The pouch may be washed and reused. The cleanness of the gel pack, which may not be machine-washable, may be protected. The gel pack may be washable by hand with soap and/or water.

The pouch may include another alternative method of closure, such as a Velcro® closure. In some embodiments, the pouch and/or the gel pack may be contoured so that it fits the perineal area, and or any other body part, such as those described above, in a comfortable fashion for a human. It should be appreciated that the pouch may fit in a snug manner over the gel pack. The snug manner may enable the gel pack to remain stationary within a threshold amount within the pouch. This may also assist in creating a comfortable-fitting apparatus for a human.

In certain embodiments, the apparatus may also be heated. The gel pack may be heated in a microwave, such as a 500-1000 Watt Microwave for approximately 30 seconds to enable the gel pack to reach a temperature of above 110 degrees Fahrenheit. The gel pack may be heated in additional increments of 10-30 seconds in order to raise the temperature of the gel pack. The gel pack may also be placed in boiling water (between about 130-212 degrees Fahrenheit) for between about 1-8 minutes.

One exemplary method for manufacturing the gel pack may be shown in Table B.

TABLE B

| Step | |
|---|---|
| Number | |
| 1. | Cutting the raw material, such as a nylon and |
| | PVC combination |
| 2. | Printing a logo (exemplary method—silk screen |
| | printing) onto the pieces of cut raw material |
| 3. | Creating the pack and leaving a small open |
| | mouth or tag |
| 4. | Mixing the gel filling, Filling the pack |
| | (exemplary method—a needle) with the gel |
| | filling, and vacuuming the pack to remove air |
| 5. | Sealing the pack using a high frequency sealing |
| | method (if a tag was used, the tag is cut off |
| | during the high frequency sealing) |
| 6. | Packing the packages to include the pack, the |
| | pouch, and any other parts |
| 7. | Random inspection of the packages |

Exemplary uses of the apparatus may include soothing and reducing swelling of the perineal area after human vaginal birth; soothing and reducing swelling of the perineal area after perineal area waxing; heat therapy for relaxing the perineal muscles during labor and delivery; cool and/or heat therapy of hemorrhoids; cool and/or heat therapy of urinary tract infections and/or yeast infections associated with the urinary area or vaginal area.

Illustrative embodiments of apparatus and methods in accordance with the principles of the invention will now be described with reference to the accompanying drawings, which form a part hereof. It is to be understood that other embodiments may be utilized and structural, functional and procedural modifications may be made without departing from the scope and spirit of the present invention.

The drawings show illustrative features of apparatus and methods in accordance with the principles of the invention. The features are illustrated in the context of selected embodiments. It will be understood that features shown in connection with one of the embodiments may be practiced in accordance with the principles of the invention along with features shown in connection with another of the embodiments.

Apparatus and methods described herein are illustrative. Apparatus and methods of the invention may involve some or all of the features of the illustrative apparatus and/or some or all of the steps of the illustrative methods. The steps of the methods may be performed in an order other than the order shown or described herein. Some embodiments may omit steps shown or described in connection with the illustrative methods. Some embodiments may include steps that are not shown or described in connection with the illustrative methods, but rather shown or described in a different portion of the specification.

One of ordinary skill in the art will appreciate that the steps shown and described herein may be performed in other than the recited order and that one or more steps illustrated may be optional. The methods of the above-referenced embodiments may involve the use of any suitable elements, steps, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

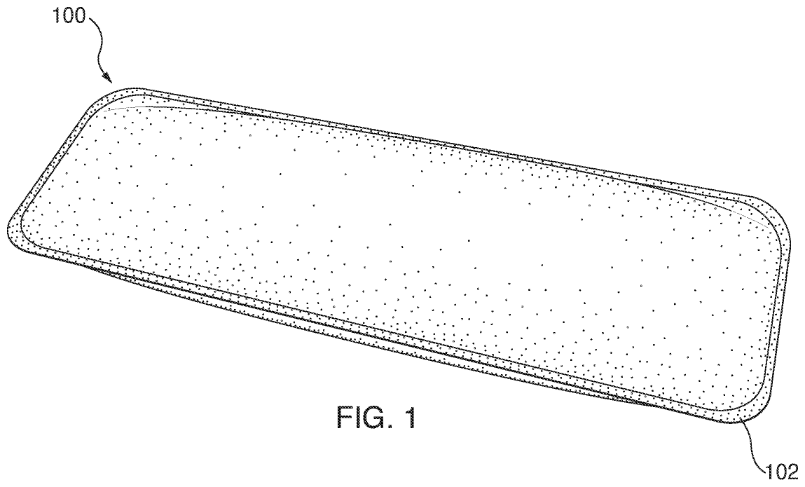

FIG. 1 shows an illustrative perspective view of gel pack 100. Gel pack 100 may be filled with a gel filling. Gel pack 100 may be sealed at the edge, as shown at 102. The edge may be about 5 millimeters wide. Gel pack 100 may be sized to fit comfortably in the perineal area.

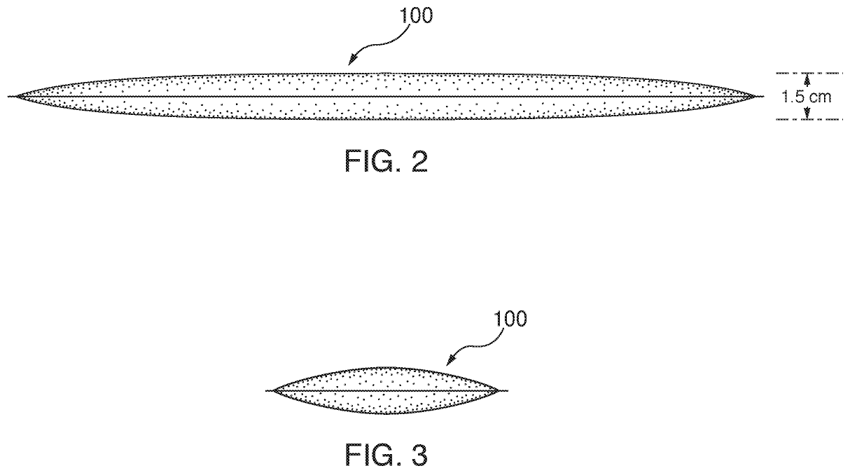

FIG. 2 shows an illustrative side view of the long side of gel pack 100. In some embodiments, the depth of gel pack 100 may be 1.5 centimeters at the deepest section. In other embodiments, the gel pack may be 1.4 centimeters to 1.6 centimeters at the deepest section.

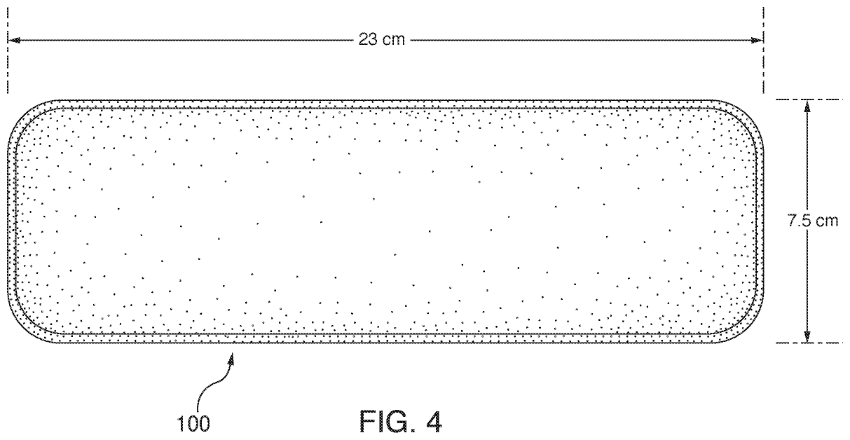

FIG. 3 shows an illustrative side view of the short side of gel pack 100. In some embodiments, the depth of gel pack 100 may be 1.5 centimeters at the deepest section. In other embodiments, the gel pack may be 1.4 centimeters to 1.6 centimeters at the deepest section.

FIG. 4 shows an illustrative top view of gel pack 100. Gel pack 100 may be about 23 centimeters long and about 7.5 centimeter wide, including the 5 millimeter edge on all sides.

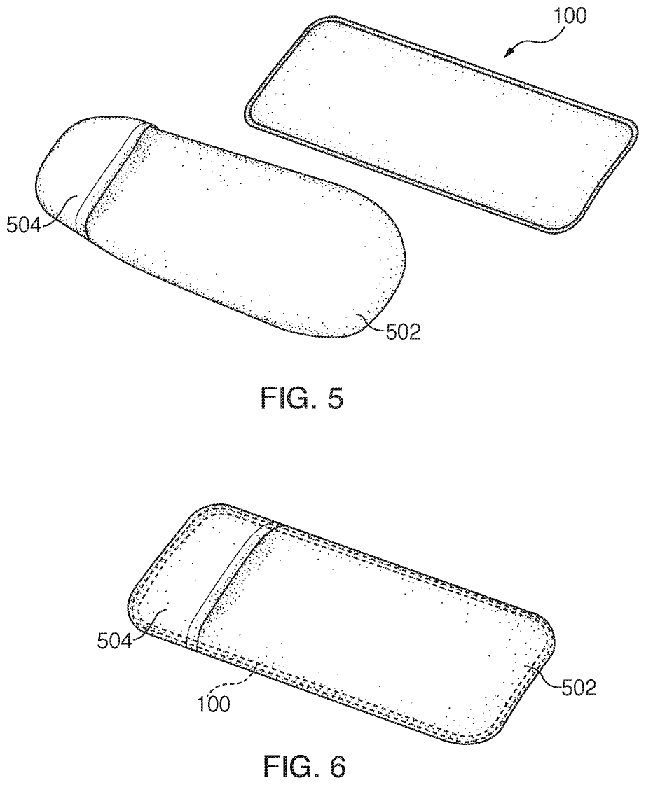

FIG. 5 shows illustrative gel pack 100 and pouch 502. Pouch 502 may include flap 504. Flap 504 may enable opening and/or closing of pouch 502.

FIG. 6 shows illustrative gel pack 100 enclosed by pouch 502. It should be appreciated that flap 504 is in a closed position to completely enclose gel pack 100 (not shown).

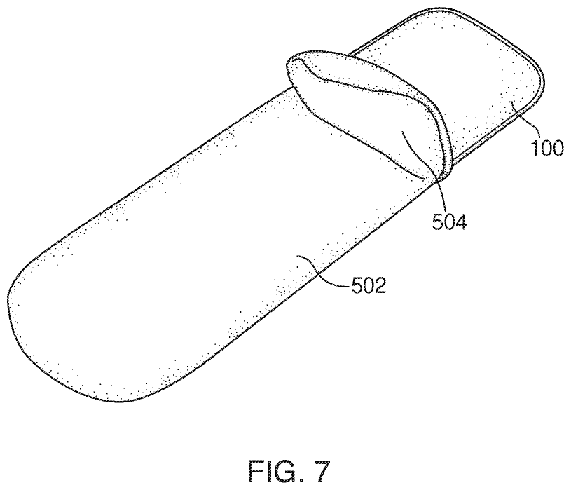

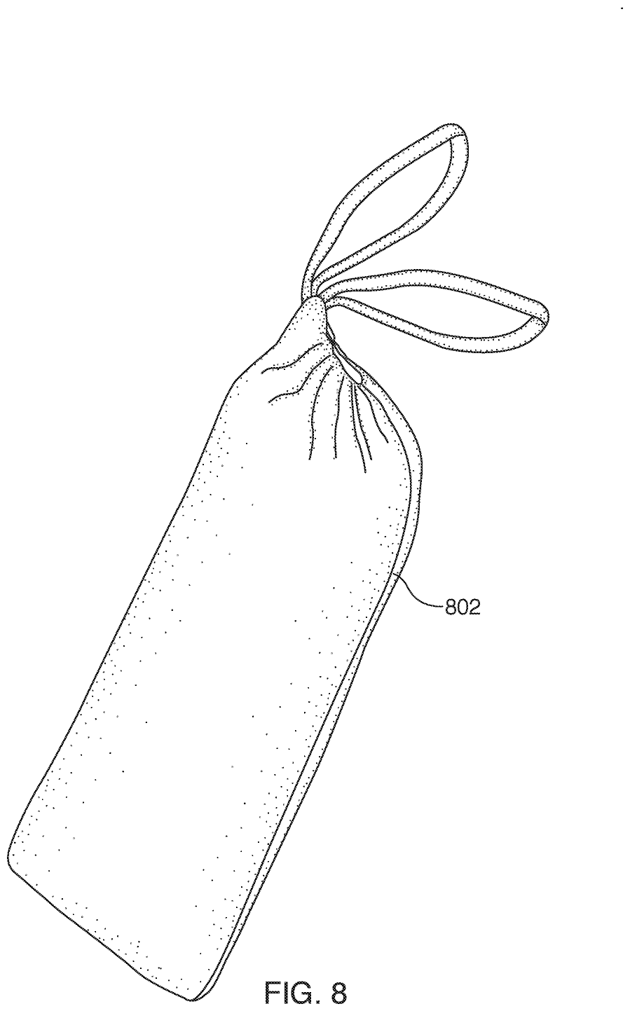

FIG. 7 shows illustrative gel pack 100 partially enclosed by pouch 502. Flap 504 may be positioned in an open position to enable gel pack 100 to be removed from pouch 502.

FIG. 8 shows illustrative drawstring bag 802. Drawstring bag 802 may weigh approximately 18 grams. Drawstring bag 802 may measure approximately 28 centimeters long and 12 centimeters wide. Drawstring bag 802 may be configured to hold three gel packs and 3 pouches.

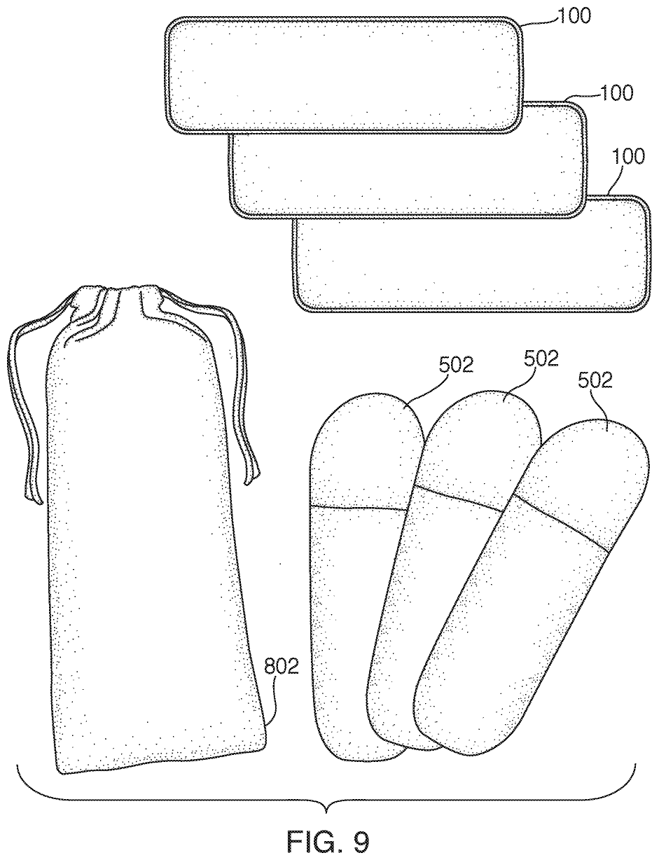

FIG. 9 shows illustrative contents of drawstring bag 802. Drawstring bag 802 may include three gel packs 100 and three pouches 502.

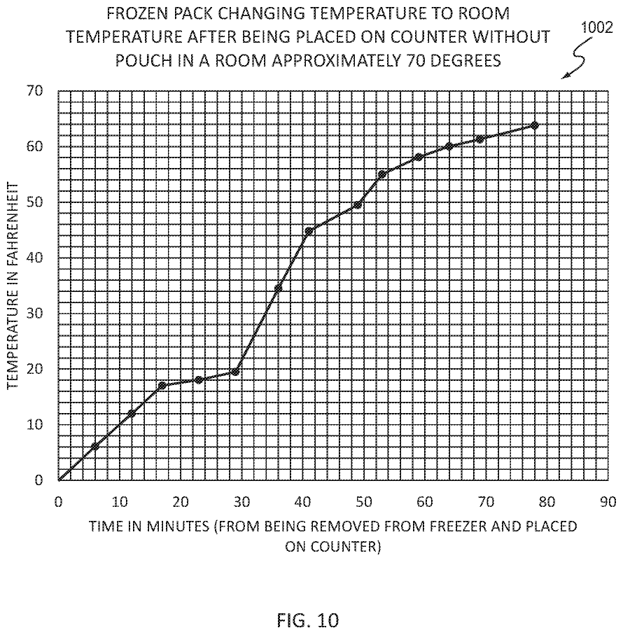

FIG. 10 shows an illustrative graph 1002. Graph 1002 shows temperature changes of a frozen gel pack changing to room temperature after being placed on a counter without a pouch in a room at approximately 70 degrees Fahrenheit. Table C shows the data from which graph 1002 was plotted.

TABLE C

| Time | Time—absolute | Temp |
|---|---|---|
| 12:06 | 0 | −0.04° F. |
| 12:12 | 6 | 6.1° F. |
| 12:18 | 12 | 12° F. |
| 12:23 | 17 | 17.04° F. |
| 12:29 | 23 | 18.06° F. |
| 12:35 | 29 | 19.5° F. |
| 12:42 | 36 | 34.5° F. |
| 12:47 | 41 | 44.8° F. |
| 12:55 | 49 | 49.5° F. |
| 12:59 | 53 | 55° F. |
| 1:05 | 59 | 58.1° F. |
| 1:10 | 64 | 60.04° F. |
| 1:15 | 69 | 61.3° F. |
| 1:24 | 78 | 63.8° F. |

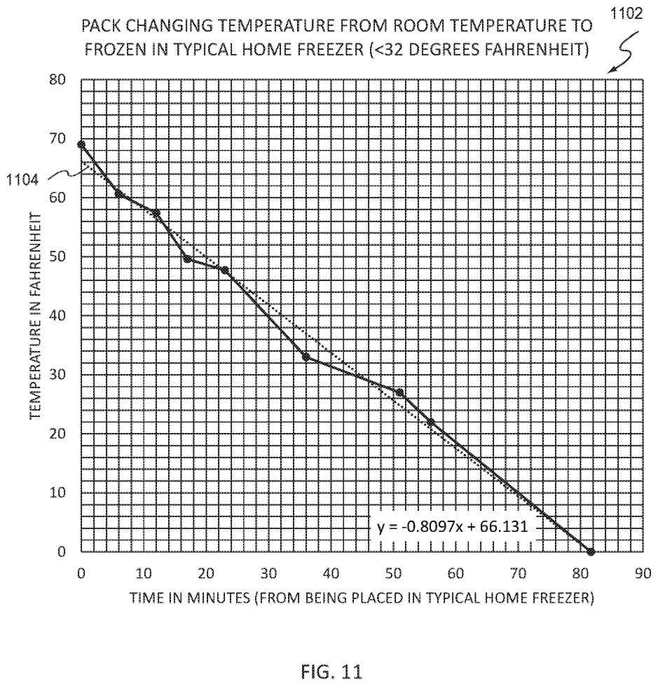

FIG. 11 shows illustrative graph 1102. Graph 1102 shows temperature of gel pack changing from room temperature to frozen temperature in a typical home freezer. Trend line 1104 is calculated. Table D shows the data from which graph 1102 was charted.

TABLE D

| Time | Time—absolute | Temp |
|---|---|---|
| 9:44 | 0 | 69° F. |
| 9:50 | 6 | 60.6° F. |
| 9:56 | 12 | 57.4° F. |
| 10:01 | 17 | 49.6° F. |
| 10:07 | 23 | 47.7° F. |
| 10:20 | 36 | 33° F. |
| 10:35 | 51 | 27° F. |
| 10:40 | 56 | 22° F. |

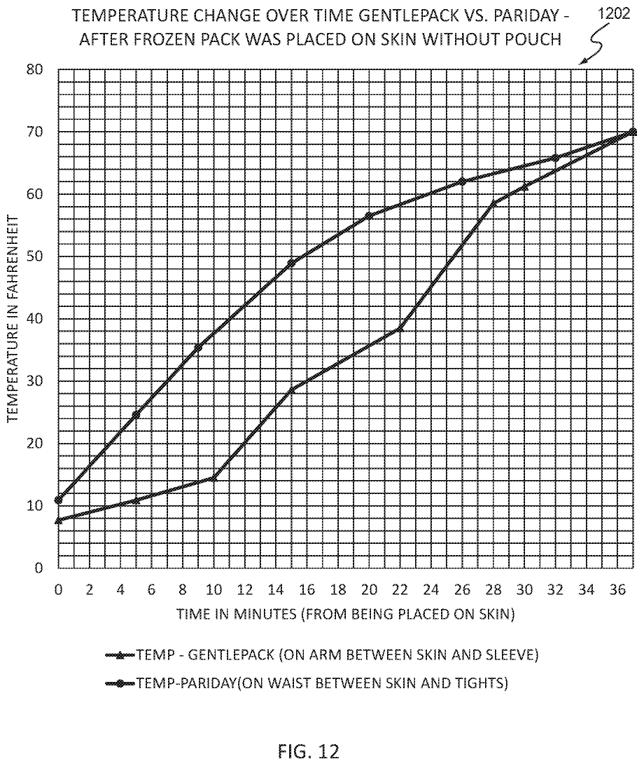

FIG. 12 shows illustrative graph 1202. Graph 1202 shows temperature changes over time comparing GentlePack™ vs. Pariday®. The GentlePack™ was placed on a human arm between the skin and the sleeve. The Pariday® was placed on a human waist between skin and stockings. Table E, F and G shows the data from which graph 1202 was charted.

TABLE E
GentlePack TM Test—on arm between skin and sleeve

| Time | Temp |
|---|---|
| 7:10 | 7.7° F. |
| 7:15 | 10.9° F. |
| 7:20 | 14.5° F. |
| 7:25 | 28.6° F. |
| 7:32 | 38.5° F. |
| 7:38 | 58.5° F. |
| 7:40 | 61.2° F. |
| 7:47 | 70° F. |

TABLE F
Pariday ® Test—on waist between skin and stockings

| Time | Temp |
|---|---|
| 4:23 | 10.9° F. |
| 4:28 | 24.6° F. |
| 4:32 | 35.4° F. |
| 4:38 | 48.9° F. |
| 4:43 | 56.5° F. |
| 4:49 | 62° F. |
| 4:55 | 65.8° F. |
| 5:00 | 70° F. |

TABLE G

| | Temp—Gentlepack | Temp—Pariday ® |
|---|---|---|
| Time- | TM (on arm between | (on waist between |
| absolute | skin and sleeve) | skin and stockings) |
| 0 | 7.7° F. | 10.9° F. |
| 5 | 10.9° F. | 24.6° F. |
| 9 | | 35.4° F. |
| 10 | 14.5° F. | |
| 15 | 28.6° F. | 48.9° F. |
| 20 | | 56.5° F. |
| 22 | 38.5° F. | |
| 26 | | 62° F. |
| 28 | 58.5° F. | |
| 30 | 61.2° F. | |
| 32 | | 65.8° F. |
| 37 | 70° F. | 70° F. |

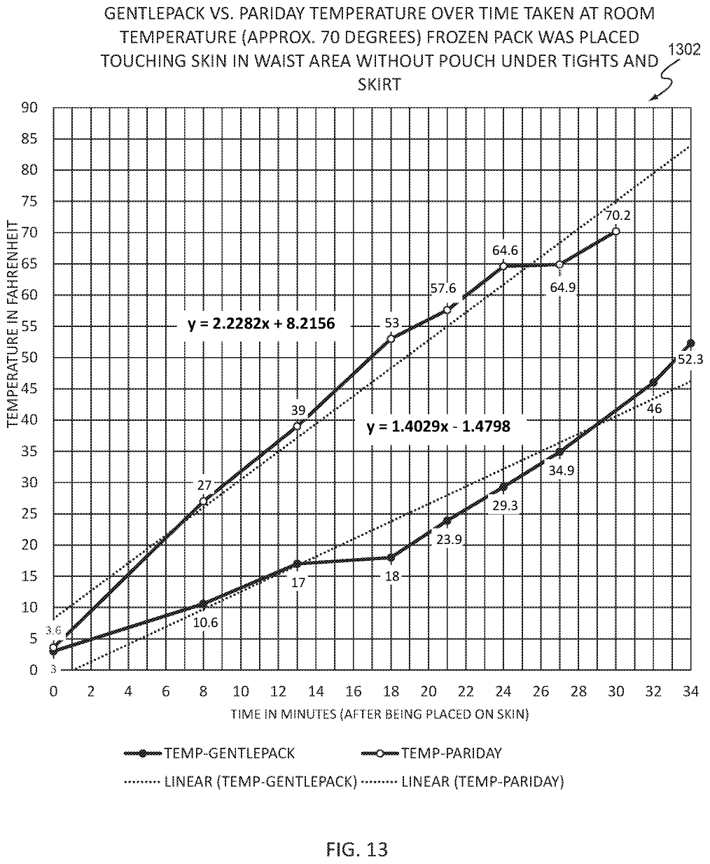

FIG. 13 shows illustrative graph 1302. Illustrative graph 1302 shows a comparison between GentlePack™ and Pariday® temperature over time. A frozen pack was placed touching skin in waist area without pouch under stockings and skirt. Trend lines shown are calculated.

Tables H, I and J show the data from which graph 1302 was charted.

TABLE H
GentlePack TM Test:

| Time | Temp |
|---|---|
| 9:12 | 3.0° F. |
| 9:20 | 10.6° F. |
| 9:25 | 17.0° F. |
| 9:30 | 18.0° F. |
| 9:33 | 23.9° F. |
| 9:36 | 29.3° F. |
| 9:39 | 34.9° F. |
| 9:44 | 46° F. |
| 9:46 | 52.3° F. |

TABLE I
Pariday ® Test:

| Time | Temp |
|---|---|
| 9:08 | 3.6° F. |
| 9:16 | 27° F. |
| 9:21 | 39.0° F. |
| 9:26 | 53° F. |
| 9:29 | 57.6° F. |
| 9:32 | 64.6° F. |
| 9:35 | 64.9° F. |
| 9:38 | 70.2° F. |

TABLE J

| Time—absolute | Temp—GentlePack TM | Temp—Pariday ® |
|---|---|---|
| 0 | 3° F. | 3.6° F. |
| 8 | 10.6° F. | 27° F. |
| 13 | 17° F. | 39° F. |
| 18 | 18° F. | 53° F. |
| 21 | 23.9° F. | 57.6° F. |
| 24 | 29.3° F. | 64.6° F. |
| 27 | 34.9° F. | 64.9° F. |
| 30 | | 70.2° F. |
| 32 | 46° F. | |
| 34 | 52.3° F. | |

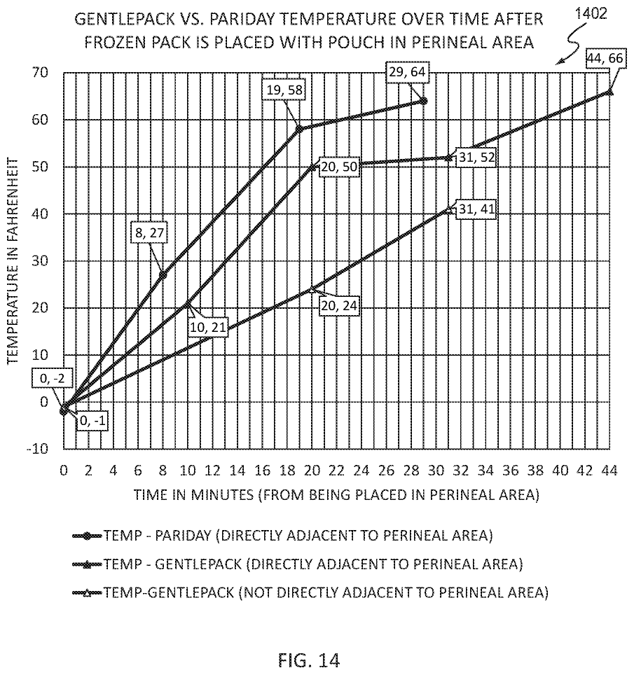

FIG. 14 shows illustrative graph 1402. Graph 1402 may compare GentlePack™ packs placed with pouch directly adjacent to perineal area and not directly adjacent to perineal area to Pariday® packs placed with pouch directly adjacent to perineal area. The temperature of the freezer used to freeze both GentlePack™ and Pariday® packs was −2° F. Tables K, L and M show data from which graph 1402 was charted.

TABLE K
Pariday ®:

| Time | Time—absolute | Temp |
|---|---|---|
| 9:36 | 0° F. | −2° F. |
| 9:44 | 8° F. | 27° F. |
| 9:55 | 19° F. | 58° F. |
| 10:05 | 29° F. | 64° F. |

TABLE L
GentlePack TM:

| | | Temp—when taken | Temp—when taken not |
|---|---|---|---|
| | Time— | directly adjacent to | directly adjacent to |
| Time | absolute | perineal area | perineal area |
| 10:28 | 0 | −1° F. | |
| 10:38 | 10 | 21° F. | |
| 10:48 | 20 | 50° F. | 24° F. |
| 10:59 | 31 | 52° F. | 41° F. |
| 11:12 | 44 | 66° F. | |

TABLE M

| | Temp— | Temp— | Temp— |
|---|---|---|---|
| | Pariday ® | GentlePack TM | GentlePack |
| | (directly | (directly | TM (not |
| Time— | adjacent to | adjacent to | directly adjacent |
| absolute | perineal area) | perineal area) | to perineal area) |
| 0 | −2° F. | −1° F. | |
| 8 | 27° F. | | |
| 10 | | 21° F. | |
| 19 | 58° F. | | |
| 20 | | 50° F. | 24° F. |
| 29 | 64° F. | | |
| 31 | | 52° F. | 41° F. |
| 44 | | 66° F. | |

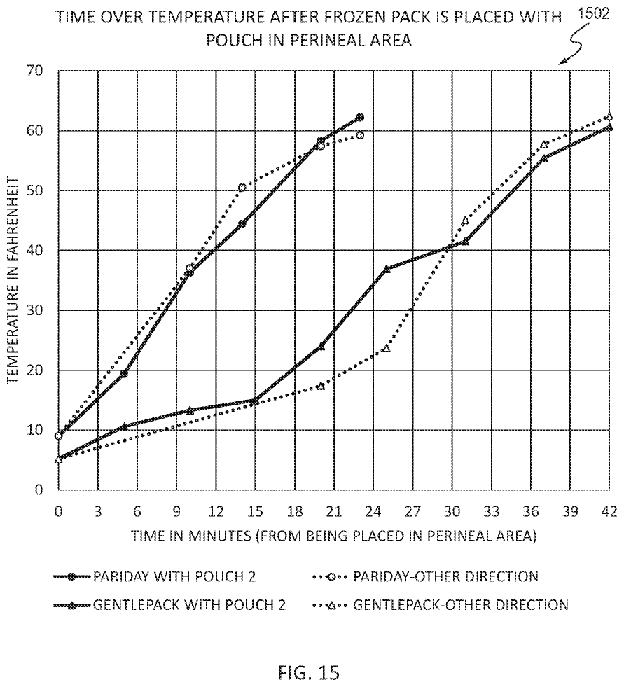

FIG. 15 shows graph 1502. Graph 1502 may show temperature changes over time after frozen GentlePack™ and Pariday® is placed with pouch is perineal area. Temperature was taken from two different places in both the Pariday® pack and the GentlePack™ pack. Table N shows data used to for graphing graph 1502.

TABLE N

| | | Temp | | | |
|---|---|---|---|---|---|
| | | Pariday ® | Pariday ® | GentlePack | GentlePack |
| | Time— | with | other | TM | TM—other |
| Time | absolute | pouch | direction | with Pouch | direction |
| 10:28 | 0 | 9° F. | 9° F. | 5.2° F. | 5.2° F. |
| 10:33 | 5 | 19.4° F. | | 10.6° F. | |
| 10:38 | 10 | 36.3° F. | 37° F. | 13.3° F. | |
| 10:42 | 14 | 44.4° F. | 50.5° F. | | |
| | 15 | | | 15° F. | |
| 10:48 | 20 | 58.3° F. | 57.4° F. | 24° F. | 17.4° F. |
| 10:51 | 23 | 62.2° F. | 59.2° F. | | |
| 11:18 | 25 | | 36.9° F. | 23.7° F. | |
| 11:24 | 31 | | 41.5° F. | 45° F. | |
| 11:30 | 37 | | 55.4° F. | 57.7° F. | |
| 11:35 | 42 | | 60.6° F. | 62.4° F. | |

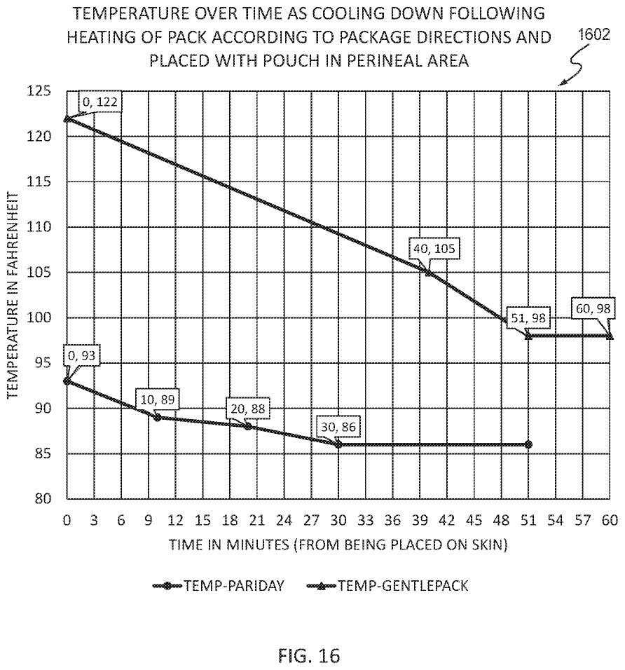

FIG. 16 shows graph 1602. Graph 1602 shows temperature changes over time as cooling down following heating of pack according to package directions. Heated pack was then placed with pouch in the perineal area. Table 0, P and Q show the data from which graph 1602 was charted.

TABLE O
Pariday ®, placed in water temperature of 110 degrees:

| Time | Temp—Pariday ® |
|---|---|
| 10:28 | 93° F. |
| 10:38 | 89° F. |
| 10:48 | 88° F. |
| 10:58 | 86° F. |
| 11:19 (approximate time) | 86° F. |

TABLE P
GentlePack TM, placed in water temperature
of 134 degrees Fahrenheit:

| Time | Temp |
|---|---|
| 11:21 | 122° F. |
| 12:01 | 105° F. |
| 12:12 | 98° F. |
| 12:21 | 98° F. |

TABLE Q

| Time—absolute | Temp—Pariday ® | Temp—GentlePack TM |
|---|---|---|
| 0 | 93° F. | 122° F. |
| 10 | 89° F. | |
| 20 | 88° F. | |
| 30 | 86° F. | |
| 40 | | 105° F. |
| 51 | 86° F. | 98° F. |
| 60 | | 98° F. |

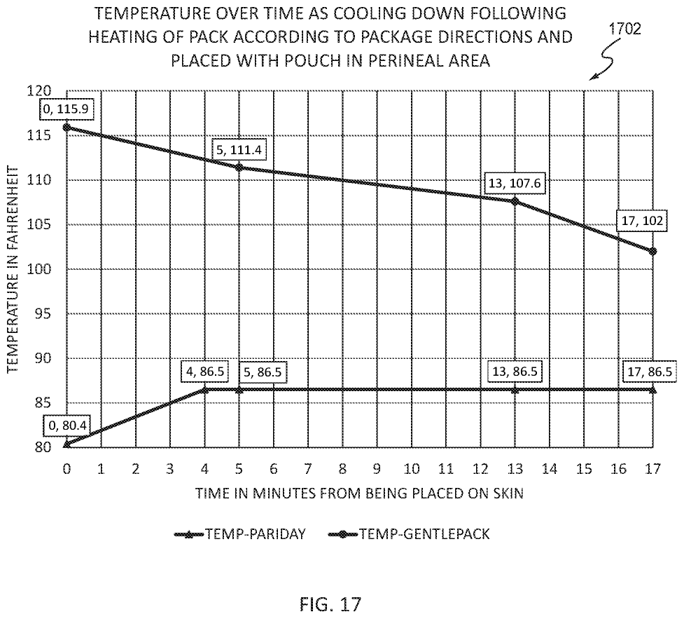

FIG. 17 shows graph 1702. Graph 1702 shows temperature changes over time as cooling down following heating of pack according to package directions. Heated pack was then placed with pouch in the perineal area. Tables R, S and T show data from which graph 1702 was charted.

TABLE R
Pariday ®

| Time | Temp—Pariday ® |
|---|---|
| 11:42 | 80.4° F. |
| 11:46 | 86.5° F. |
| 11:47 (approximated) | 86.5° F. |
| 11:55 (approximated) | 86.5° F. |
| 11:59 (approximated) | 86.5° F. |

TABLE S
GentlePack TM, placed in water temperature 170.4:

| Time | Temp—GentlePack TM |
|---|---|
| 11:51 | 115.9° F. |
| 11:56 | 111.4° F. |
| 12:04 | 107.6° F. |
| 12:08 | 102° F. |

TABLE I

| Time—absolute | Temp—Pariday ® | Temp—GentlePack TM |
|---|---|---|
| 0 | 80.4° F. | 115.9° F. |
| 4 | 86.5° F. | |
| 5 | 86.5° F. | 111.4° F. |
| 13 | 86.5° F. | 107.6° F. |
| 17 | 86.5° F. | 102° F. |

It should be appreciated that the results shown above were unexpected based on prior knowledge and expectations.

Thus, methods and apparatus for a hot and cold pack with a closable pouch are provided. Persons skilled in the art will appreciate that the present invention can be practiced by other than the described embodiments, which are presented for purposes of illustration rather than of limitation, and that the present invention is limited only by the claims that follow.

What is claimed is:

1. An apparatus comprising:
a gel filling, said gel filling between about 110-250 grams, said gel filling comprising:
10-20% glycerin;
70-80% carboxymethyl cellulose (“CMC”); and
5-15% water;
a top cover and a bottom cover, said top cover and bottom cover being sealed together to form a sealed gel pack, using a high-frequency sealing method, said gel pack containing the gel filling, said top cover and bottom cover measuring between about 22-26 centimeters long and between about 6.5-8.5 centimeters wide, when measured flat;
wherein, when the gel pack is frozen to a temperature of less than twelve degrees Fahrenheit, the gel filling maintains a temperature under 60 degrees Fahrenheit after being placed in skin contact for a minimum of 27 minutes.

2. The apparatus of claim 1, wherein the top cover and the bottom cover are comprised of a combination of nylon and polyvinyl chloride (“PVC”).

3. The apparatus of claim 1, wherein the gel filling is between about 661.10735481932 and about 1,202.0133723988 milligrams per centimeter squared.

4. The apparatus of claim 1, further comprising a closable pouch for partial and/or total enclosure of the gel pack.

5. The apparatus of claim 4, wherein the pouch is comprised of cotton and polyester blend terry cloth fabric.

6. The apparatus of claim 5, wherein the cotton percentage, included in the terry cloth fabric, is between about 70-100 percent and the polyester percentage, included in the terry cloth fabric, is between about 0-30 percent.

7. The apparatus of claim 6, wherein a thickness of the terry cloth fabric is between about 100-300 grams per square meter (“gsm”).

8. The apparatus of claim 7, wherein the thickness of the terry cloth fabric is between about 150-200 gsm.

9. The apparatus of claim 4, wherein the pouch and/or the gel pack is partially and/or completely the color black.

10. The apparatus of claim 4, wherein the pouch comprises a flap, whereby in a closed position the pouch completely encloses the gel pack.

11. The apparatus of claim 4, wherein the pouch comprises a flap, whereby in a closed position the pouch partially encloses the gel pack.

12. The apparatus of claim 1, wherein when the gel pack is heated to above 110 degrees Fahrenheit, the gel filling maintains a temperature above 100 degrees Fahrenheit after at least fifteen minutes of skin contact.

13. An apparatus comprising:
a gel filling, said gel filling measuring between about 110 grams-250 grams;
a gel pack, said gel pack containing the gel filling, said gel pack comprising:
a top cover, said top cover:
being comprised of nylon and polyvinyl chloride; and
measuring between about 22-26 centimeters long and between about 6.5-8.5 centimeters wide, when measured flat;
a bottom cover, said bottom cover:
being comprised of a nylon and polyvinyl chloride (“PVC”);
measuring between about 22-26 centimeters long and between about 7-8 centimeters wide, when measured flat;
wherein, when the gel pack is frozen to a temperature of less than twelve degrees Fahrenheit, the gel filling will maintain a temperature under sixty degrees Fahrenheit after being placed in skin contact for a minimum of 27 minutes.

14. A perineal-sized apparatus comprising:
a gel filling;
a gel pack, said gel pack for containing the gel filling; and
a closable pouch for partial and/or total enclosure of the gel pack;
wherein, when the perineal-sized pack is frozen to at least less than about twelve degrees Fahrenheit, the gel filling maintains a temperature under 60 degrees Fahrenheit after being placed in skin contact for a minimum of 27 minutes.

15. The apparatus of claim 14, wherein the pouch is machine washable.

16. The apparatus of claim 14, wherein the pouch is disposable.

17. The apparatus of claim 16, wherein the disposable pouch is constructed of polypropylene fabric.

18. The apparatus of claim 14, wherein the pack is hand washable with soap and water.

19. The apparatus of claim 14, wherein the pack and/or the pouch is contoured to fit a human perineal area.

20. The apparatus of claim 14, wherein the pouch is microwavable.

21. The apparatus of claim 14, wherein the pouch is heated by being placed in hot water.
